(19) United States
(12) Patent Application Publication
(10) Pub. No.: US 2024/0402464 A1
(43) Pub. Date: Dec. 5, 2024

(54) OPHTHALMIC LENS

(71) Applicant: Young Optics Inc., Hsinchu Science Park (TW)

(72) Inventors: CHING-SHENG CHANG, Hsinchu Science Park (TW); CHENG-HUNG CHEN, Hsinchu Science Park (TW); CHING-LUN LIN, Hsinchu Science Park (TW)

(21) Appl. No.: 18/662,408

(22) Filed: May 13, 2024

(30) Foreign Application Priority Data
May 30, 2023 (TW) 112120120

(51) Int. Cl. G02B 9/62; G02B 7/02; G02B 9/64

(52) U.S. Cl. CPC G02B 9/62; G02B 7/023; G02B 9/64

(57) ABSTRACT

An ophthalmic lens includes a first lens group having a positive refractive power, a second lens group having a positive refractive power, and a third lens group having a positive refractive power. The first lens group includes a first lens, and a lens in the second lens group closest to the first lens group is a second lens. The ophthalmic lens satisfies the condition of 0.65<R1/R2<7.69, where R1 is a shortest distance from a central axis of an optical surface of the first lens to an outermost turning point of the optical surface of the first lens, and R2 is a shortest distance from a central axis of an optical surface of the second lens to an outermost turning point of the optical surface of the second lens.

### CROSS-REFERENCE TO RELATED APPLICATION

This application claims the priority benefit of Taiwan application serial no. 112120120, filed May 30, 2023. The entirety of the above-mentioned patent application is hereby incorporated by reference herein and made a part of this specification.

### BACKGROUND

### Field of the Invention

The invention relates to an imaging lens, more particularly to an ophthalmic lens capable of capturing images of the human fundus or other organs.

### Description of the Related Art

Existing designs of ophthalmic lenses face challenges in achieving a balance between wide viewing angles, minimal ghosting, large apertures, reduced number of lenses, lightness, and high resolution. Consequently, there is a pressing demand to provide an ophthalmic lens having superior imaging quality that satisfies these requirements and can be coupled with a mobile device or various lens modules.

### BRIEF SUMMARY OF THE INVENTION

In order to achieve one or a portion of or all of the objects or other objects, one embodiment of the invention provides an ophthalmic lens includes a first lens group having a positive refractive power, a second lens group having a positive refractive power, and a third lens group having a positive refractive power. The first lens group includes a first lens, and the first lens is an aspheric lens. The second lens group includes three lenses, one of the three lenses is an aspheric lens, a lens in the second lens group closest to the first lens group is a second lens, and the three lenses includes two closely adjacent surfaces having substantially the same curvatures. The third lens group includes two lenses, and one of the two lenses is an aspheric lens. An aperture stop is disposed between the second lens group and the third lens group, and a total number of lenses with refractive powers of the ophthalmic lens ranges from 6 to 9. The ophthalmic lens satisfies the following conditions: 0.36<L1/LT<1.12, where L1 is a distance between the first lens group and the second lens group measured along an optical axis of the ophthalmic lens, and LT is a distance measured along the optical axis between two outermost lens surfaces at opposite ends of the ophthalmic lens; 0.65<R1/R2<7.69, where R1 is a shortest distance from a central axis of an optical surface of the first lens to an outermost turning point of the optical surface of the first lens, and R2 is a shortest distance from a central axis of an optical surface of the second lens to an outermost turning point of the optical surface of the second lens; and 14.18<|LT/EFL|<41.69, where EFL is an effective focal length of the ophthalmic lens.

Another embodiment of the invention provides an ophthalmic lens includes a first lens group having a positive refractive power, a second lens group having a positive refractive power, and a lens module interface for coupling the ophthalmic lens with a lens module or a smartphone. The first lens group includes a first lens, and the first lens is an aspheric lens. The second lens group includes an aspheric lens and a cemented lens. The ophthalmic lens satisfies the following condition: 0.65<R1/R2<7.69, where R1 is a shortest distance from a central axis of an optical surface of the first lens to an outermost turning point of the optical surface of the first lens, and R2 is a shortest distance from a central axis of an optical surface of the second lens to an outermost turning point of the optical surface of the second lens.

According to the above embodiments, an ophthalmic lens with at least one of the advantages of a wide field of view, low ghost imaging, a large aperture, a reduced number of lenses, a wide diopter range, and high resolution visible/near-infrared imaging can be provided. Additionally, a high imaging quality ophthalmic lens that can be coupled with a mobile device or other lens module is also provided.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 shows a schematic diagram of an ophthalmic lens according to a first embodiment of the invention.

FIG. 2A, FIG. 2B and FIG. 2C respectively show longitudinal spherical aberration curves, astigmatic field curves and a distortion curve of the ophthalmic lens illustrated in FIG. 1 measured at wavelengths of 650 nm, 555 nm, and 450 nm.

FIG. 3 shows a schematic diagram of an ophthalmic lens according to a second embodiment of the invention.

FIG. 4A, FIG. 4B and FIG. 4C respectively show longitudinal spherical aberration curves, astigmatic field curves and a distortion curve of the ophthalmic lens illustrated in FIG. 3 measured at wavelengths of 650 nm, 555 nm, and 450 nm.

FIG. 5 shows a schematic diagram of an ophthalmic lens according to a third embodiment of the invention.

FIG. 6A, FIG. 6B and FIG. 6C respectively show longitudinal spherical aberration curves, astigmatic field curves and a distortion curve of the ophthalmic lens illustrated in FIG. 5 measured at wavelengths of 650 nm, 555 nm, and 450 nm.

FIG. 7 shows a schematic diagram of an ophthalmic lens according to a fourth embodiment of the invention.

FIG. 8A, FIG. 8B and FIG. 8C respectively show longitudinal spherical aberration curves, astigmatic field curves and a distortion curve of the ophthalmic lens illustrated in FIG. 7 measured at wavelengths of 650 nm, 555 nm, and 450 nm.

FIG. 9 shows a schematic diagram of an ophthalmic lens according to a fifth embodiment of the invention.

FIG. 10A, FIG. 10B and FIG. 10C respectively show longitudinal spherical aberration curves, astigmatic field curves and a distortion curve of the ophthalmic lens illustrated in FIG. 9 measured at wavelengths of 650 nm, 555 nm, and 450 nm.

FIG. 11 shows a schematic diagram of an ophthalmic lens according to a sixth embodiment of the invention.

FIG. 12A, FIG. 12B and FIG. 12C respectively show longitudinal spherical aberration curves, astigmatic field curves and a distortion curve of the ophthalmic lens illustrated in FIG. 11 measured at wavelengths of 650 nm, 555 nm, and 450 nm.

FIG. 13 shows a schematic diagram of a relay ophthalmic lens coupled with a smart phone according to an embodiment of the invention.

FIG. 14 shows a schematic diagram of an ophthalmic lens having a diopter adjustment mechanism according to an embodiment of the invention.

FIG. 15 shows a schematic diagram of an ophthalmic lens according to another embodiment of the invention.

FIG. 16 shows a schematic diagram of an ophthalmic lens according to another embodiment of the invention.

### DETAILED DESCRIPTION OF THE INVENTION

In the following detailed description of the preferred embodiments, directional terminology, such as “top,” “bottom,” “front,” “back,” etc., is used with reference to the orientation of the Figure(s) being described. The components of the invention can be positioned in a number of different orientations. As such, the directional terminology is used for purposes of illustration and is in no way limiting. Further, “First,” “Second,” etc., as used herein, are used as labels for nouns that they precede, and do not imply any type of ordering (e.g., spatial, temporal, logical, etc.).

The term “lens” refers to an element made from a partially or entirely light-penetrable material with optical power. The material commonly includes plastic or glass.

In an imaging system, an object side may refer to one side of an optical path of an ophthalmic lens comparatively near a subject (human eyes) to be picked-up, and an image side may refer to other side of the optical path comparatively near a photosensor.

A certain region of an object side surface (or an image side surface) of a lens may be convex or concave. Herein, a convex or concave region is more outwardly convex or inwardly concave in the direction of an optical axis as compared with other neighboring regions of the object/image side surface.

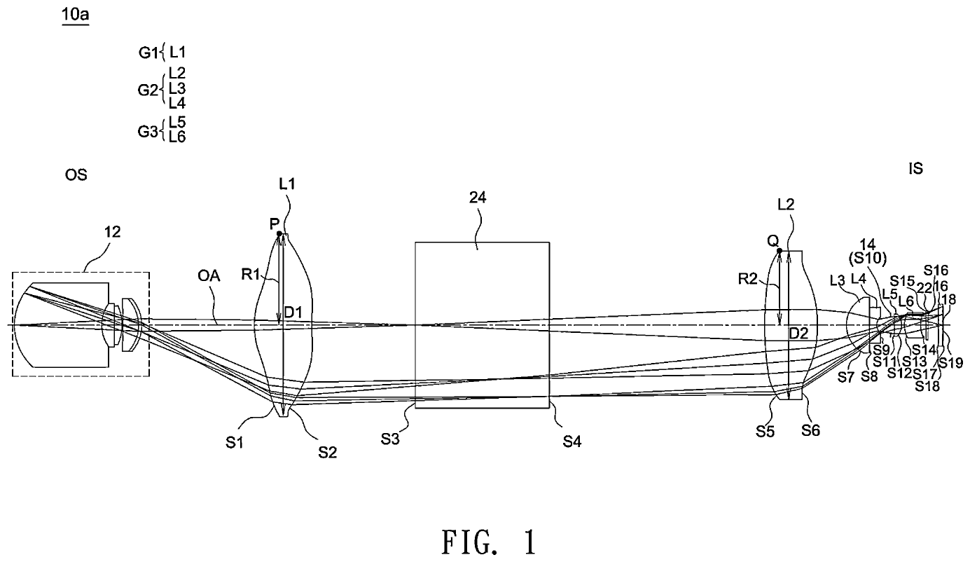

FIG. 1 shows a schematic diagram of an ophthalmic lens according to a first embodiment of the invention. The ophthalmic lens may serve as an imaging lens for capturing images of human organs such as the fundus, the inner ear, or the skin. Referring to FIG. 1, the ophthalmic lens 10a includes a lens group G1 with a positive refractive power, a lens group G2 with a positive refractive power, and a lens group G3 with a positive refractive power arranged in order along a direction. In this embodiment, the lens group G1 includes a lens L1, the lens group G2 include a lens L2, a lens L3 and a lens L4, and the lens group G3 includes a lens L5 and a lens L6. In this embodiment, the ophthalmic lens 10a is housed within a barrel (not shown) and consists essentially of six lenses, namely the lenses L1, L2, L3, L4, L5 and L6 arranged in order from an object side OS to an image side IS of the ophthalmic lens 10a, where the object side OS corresponds to the position of an eye model 12 and the image side IS corresponds to the position of an image plane 18 of an image sensor. In this embodiment, the refractive powers of the lenses L1 to L6 along the optical axis OA are respectively positive, positive, positive, negative, positive and positive. The lens L1, the lens L2, and the lens L6 are aspheric lenses and may be made of plastic materials, such as polymethyl methacrylate (PMMA) or polycarbonate (PC), and the lens L3, the lens L4, and the lens L5 are glass spherical lenses, but the invention is not limited thereto. In this embodiment, the three lenses of the lens group G2 may include two paired surfaces that are closely adjacent each other and have substantially the same curvatures (such as surfaces respectively on the lens L3 and the lens L4 that are closely adjacent and directly face each other). In this embodiment, the lens L3 and the lens L4 are paired together, such as being cemented to each other, to form a doublet lens to enhance the chromatic aberration correction and permit more relaxed tolerances in manufacturing the ophthalmic lens 10a to thus enhance production yield. Furthermore, a cover plate 16 and an image sensor (not shown) can be arranged on the image side IS. The cover plate 16 may be a plate made of any suitable light-penetrable material, such as glass. The cover plate 16 may function to adjust the optical path length and protect the ophthalmic lens. An image plane of the ophthalmic lens 10a on the image sensor is marked as 18. In this embodiment, the ophthalmic lens 10a may perform focusing operations using near-infrared (NIR) light before capturing images. Subsequently, by moving the image sensor within a certain range based on prescribed focal plane differences between white light and NIR, it is possible to directly capture images in a white light environment, without the need of refocusing for white light imaging. The aperture stop 14 is a light-blocking element that limits the amount of light passing through the ophthalmic lens 10a. In other embodiment, the aperture stop 14 can be defined by an inner diameter of a lens barrel and thus is not an independent optical element. In this embodiment, a filter 22 is placed between the lens group G3 and the cover plate 16 to allow light within a specific wavelength range (such as visible light or near-infrared light) to pass therethrough. In other embodiment, the filter 22 may be replaced by a coating or omitted from the ophthalmic lens 10a. Furthermore, in this embodiment, a light deflecting element 24, such as a polarizing beam splitter (PBS) or a dichroic mirror, is arranged between the lens group G1 and the lens group G2 in an illumination optical path of the ophthalmic lens 10a. In other embodiment, the light deflecting element 24 may be disposed in the lens group G2 or between the lens group G2 and the lens group G3. The light deflecting element 24 may reflect light beams from a fixation light (not shown) or reflect near-infrared light used for focusing, allowing the image sensor and the fixation light to be positioned at an equivalent focal plane of the ophthalmic lens. In this embodiment, the light beam reflected by the human eye model 12 sequentially passes through the lens L1, the light deflecting element 24, the lens L2, the lens L3, the lens L4, the aperture stop 14, the lens L5, the lens L6 and the cover plate 16 and then forms an image on the image plane 18. In this embodiment, the ophthalmic lens 10a is a dual-stage imaging system, where the primary imaging occurs between the lens group G1 and the lens group G2, while the secondary imaging takes place at the image plane 18.

In at least some embodiments of the invention, the lens group G1 may include an aspheric lens with a refractive power, and the lens group G2 may include three lenses with refractive powers. One of the three lenses of the second lens group G2 is an aspheric lens, and the other two lenses form a cemented doublet. The lens group G3 may include two lenses with refractive powers, and one of the two lenses is an aspheric lens. The lens closest to the object side OS may be a plano-convex lens or a meniscus lens, and the convex surface in the paraxial region of the plano-convex lens or the meniscus lens faces the object side OS. Moreover, the lens closest to the image side IS may be a meniscus lens, and the concave surface in the paraxial region of the meniscus lens faces the image side IS. This arrangement may reduce the total number of lenses as required. Herein, the “paraxial region” can be defined as the surface area of a lens between the optical axis and a turning point of the lens closest to the optical axis. Moreover, in at least some embodiments of this invention, a distance measured along the optical axis OA from the lens surface closest to the object side OS to the aperture stop 14 is greater than a distance measured along the optical axis OA from the lens surface closest to the image side IS to the aperture stop 14. In at least some embodiments of this invention, a distance between the lens group G1 and the lens group G2 can be greater than a distance between the lens group G2 and the lens group G3, and a distance between the lens group G1 and the lens group G2 can be more than twice a length of the lens group G2 measured along the optical axis OA.

According to various embodiments of the invention, the number of lenses with refractive powers of an ophthalmic lens ranges from 6 to 9, but the number, shape and optical characteristic of lenses can be designed according to actual needs without limitation. For example, in one embodiment, a lens with a comparatively larger thickness can be replaced with two singlet lenses that are stacked together by a spacing of less than 0.05 mm and have substantially the same radius of curvature in two adjacent lens surfaces, and the two singlet lenses may respectively have a high Abbe number and a low Abbe number to facilitate chromatic aberration corrections and hence improve imaging resolution.

In each of the following embodiments, the object side OS is located on the left side and the image side IS is located on the right side of each figure, and thus this is not repeatedly described in the following for brevity.

Detailed optical data and design parameters of the ophthalmic lens 10a are shown in Table 1 below. Note the data provided below are not used for limiting the invention, and those skilled in the art may suitably modify parameters or settings of the following embodiment with reference of the invention without departing from the scope or spirit of the invention.

Table 1 lists the values of parameters for each lens of the ophthalmic lens 10a, where the surface symbol denoted by an asterisk is an aspherical surface. Besides, the radius of curvature, interval and diameter shown in Table 1 are all in a unit of mm.

TABLE 1

| | | Radius of | Interval | Refractive | Abbe |
|---|---|---|---|---|---|
| Object description | Surface | curvature(mm) | (mm) | index (nd) | number (Vd) |
| eye model 12 | | | 21.34 | | |
| lens L1(aspheric) | S1* | 14.38 | 11.000 | 1.54 | 56.3 |
| | S2* | 250.00 | 59.443 | | |
| deflecting element | S3 | Inf. | 25.400 | 1.52 | 64.2 |
| 24 | | | | | |
| | S4 | Inf. | 1.000 | | |
| lens L2(aspheric) | S5* | 92.15 | 9.999 | 1.64 | 23.3 |
| | S6* | −16.24 | 5.262 | | |
| lens L3(meniscus) | S7 | 5.85 | 4.388 | 1.83 | 42.7 |
| lens L4(meniscus) | S8 | 71.52 | 1.290 | 1.99 | 16.5 |
| | S9 | 2.00 | 2.435 | | |
| aperture stop 14 | S10 | Inf. | 0.918 | | |
| lens L5(meniscus) | S11 | −37.09 | 1.436 | 1.50 | 81.6 |
| | S12 | −3.19 | 0.554 | | |
| lens L6(aspheric) | S13* | 4.40 | 3.000 | 1.54 | 56.3 |
| | S14* | 8.72 | 1.146 | | |
| filter 22 | S15 | Inf. | 0.210 | 1.52 | 64.2 |
| | S16 | Inf. | 2.000 | | |
| cover plate 16 | S17 | | 0.500 | 1.52 | 64.2 |
| | S18 | Inf. | 0.045 | | |
| image plane 18 | S19 | Inf. | 0.000 | | |

In the above Table 1, the field heading “interval” represents a distance between two adjacent optical surfaces along the optical axis OA. For example, an interval of the surface S1 is a distance between the surface S1 and the surface S2 along the optical axis OA. Further, the interval, refractive index and Abbe number of any lens listed in the column of “Object description” show values in a horizontal row aligned with the position of that lens. Moreover, in table 1, the surfaces S1 and S2 are respectively the object-side surface and image-side surface of the lens L1, the surfaces S5 and S6 are respectively the object-side surface and image-side surface of the lens L2, and the remaining lens surfaces are classified by analogy so that related descriptions are omitted for sake of brevity.

The radius (radius of curvature) in the above table is a reciprocal of the curvature. When a lens surface has a positive radius of curvature, the center of the lens surface is located towards the image side. When a lens surface has a negative radius of curvature, the center of the lens surface is located towards the object side.

In at least some embodiments of the invention, the ophthalmic lens may satisfy a condition of 0.36<L1/LT<1.12, and more preferably 0.55<L1/LT<0.75, where L1 is a distance between the lens group G1 and the lens group G2 along the optical axis OA of the ophthalmic lens, and LT denotes a total lens length that is a distance measured along the optical axis OA between two outermost lens surfaces at opposite ends of the ophthalmic lens (such as surfaces S1 and S14 shown in FIG. 1). Furthermore, in at least some embodiments of the invention, the ophthalmic lens may satisfy a condition of 14.18<|LT/EFL|<41.69, and more preferably 21.27<|LT/EFL|<27.80, where EFL is an effective focal length of the ophthalmic lens. Additionally, the ophthalmic lens may satisfy a condition of 4.3<|EFL1/EFL|<5.83, where EFL1 is an effective focal length of the lens group closest to the object side OS (such as the lens group G1). In this embodiment of the ophthalmic lens 10a, L1=85.8 mm, LT=126.1 mm, L1/LT=0.68, EFL=−5.15 mm, EFL1=27.48 mm, |LT/EFL|=24.47, and |EFL1/EFL|=5.336.

In at least some embodiments of this invention, the ophthalmic lens may satisfy a condition of 0.65<R1/R2<7.69, preferably 0.65<R1/R2<2.42, and more preferably 0.98<R1/R2<1.61. As shown in FIG. 1, in the ophthalmic lens 10a, the lens L1 is closest to the object side OS (human eye model 12), and the lens L2 is next closest to the object side OS and is closest to the lens group G1 among all lenses of the lens group G2. The outermost turning point (farthest from the optical axis OA) of an optical surface of the lens L1 is denoted as P, and an outermost turning point of an optical surface of the lens L2 is denoted as Q. R1 is a shortest distance measured from the outermost turning point P of the lens L1 to a central axis of the optical surface of lens L1 (the distance measured in the direction perpendicular to the optical axis OA), and R2 is a shortest distance measured from the outermost turning point Q of the lens L2 to a central axis of the optical surface of the lens L2, where the distances R1 and R2 can also be considered as the radii of the optical surfaces. In this embodiment of the ophthalmic lens 10a, R1/R2=1.23. Furthermore, the ophthalmic lens may satisfy a condition of 0.65<D1/D2<2.42, where D1 is a diameter of an optical surface of the lens L1, and D2 is a diameter of an optical surface of the lens L2. Herein, the “optical surface” mentioned in the above refers to the part of a lens surface not covered by a lens flange, and the diameter of the optical surface is greater than a clear aperture (CA). More specifically, the diameter D1 is the greater diameter among the object-side optical surface and the image-side optical surface of the lens L1, and the diameter D2 is the greater diameter among the object-side optical surface and the image-side optical surface of the lens L2. Meeting the condition of 0.65<D1/D2<2.42 is beneficial for appropriately converging the light entering the ophthalmic lens to achieve better optical effects within a limited space. In this embodiment of the ophthalmic lens 10a, the D1=32.28 mm, D2=26.25 mm, and D1/D2=1.230.

A diagonal field of view (DFOV) refers to a light collection angle of the optical surface closest to the object side OS; that is, the DFOV is a full field of view measured diagonally. In at least some embodiments of the invention, the DFOV is greater than 30 degrees and smaller than 60 degrees. In this embodiment, the DFOV of the ophthalmic lens 10a is 45 degrees. In at least some embodiments of the invention, an F-number (F #) of the ophthalmic lens is greater than 1.6 and smaller than 2.8. In this embodiment, an F-number of the ophthalmic lens 10a is 2.0.

An aspheric lens indicates at least one of its front lens surface and rear lens surface has a radius of curvature that varies along a center axis to correct abbreviations. In the following design examples of the invention, each aspheric surface satisfies the following equation:

$$Z = \frac{{cr}^{2}}{1 + \sqrt{1 - ( 1 + k ) {c}^{2} {r}^{2}}} + {Ar}^{4} + {Br}^{6} + {Cr}^{8} + {Dr}^{10} + {Er}^{12} + {Fr}^{14} + {Gr}^{16} + … ,$$

where Z denotes a sag of an aspheric surface along the optical axis OA, c denotes a reciprocal of a radius of an osculating sphere, K denotes a conic constant, r denotes a height of the aspheric surface measured in a direction perpendicular to the optical axis OA, and parameters A-G are 4th, 6th, 8th, 10th, 12th, 14th and 16th order aspheric coefficients. Note the data provided below are not used for limiting the invention, and those skilled in the art may suitably modify parameters or settings of the following embodiment with reference of the invention without departing from the scope or spirit of the invention.

TABLE 2

| Surface | S1* | S2* | S5* | S6* | S13* | S14* |
|---|---|---|---|---|---|---|
| K | −0.48 | 0.00 | 2.42 | 0.00 | −0.34 | 0.00 |
| A | −1.66E−04 | −2.43E−04 | 5.70E−05 | 1.48E−04 | 1.06E−03 | 2.99E−03 |
| B | 3.35E−07 | 2.18E−06 | −5.45E−07 | −9.82E−07 | 3.00E−05 | 6.98E−04 |
| C | −1.34E−09 | −1.82E−08 | 2.62E−09 | 4.77E−09 | −9.34E−06 | −5.89E−05 |
| D | 1.08E−11 | 1.09E−10 | 5.27E−12 | 5.06E−11 | 2.60E−07 | −1.17E−07 |
| E | −5.53E−14 | −4.19E−13 | −1.07E−13 | −8.65E−13 | 2.66E−11 | −6.93E−09 |
| F | 1.54E−16 | 9.27E−16 | 5.79E−16 | 5.06E−15 | −8.83E−21 | −1.52E−11 |
| G | −1.82E−19 | −8.74E−19 | −1.03E−18 | −1.01E−17 | 0.00E+00 | −1.51E−15 |

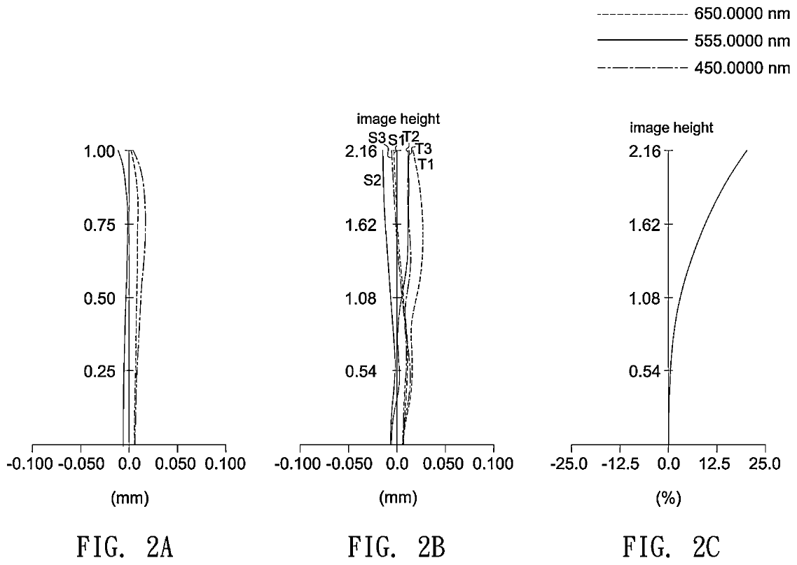

FIG. 2A, FIG. 2B and FIG. 2C show longitudinal spherical aberration curves, astigmatic field curves and a distortion curve of the ophthalmic lens 10a measured at wavelengths of 650 nm, 555 nm, and 450 nm. The simulation results shown in FIGS. 2A-2C are within permitted ranges specified by the standard, which indicates the ophthalmic lens 10a may provide good imaging quality.

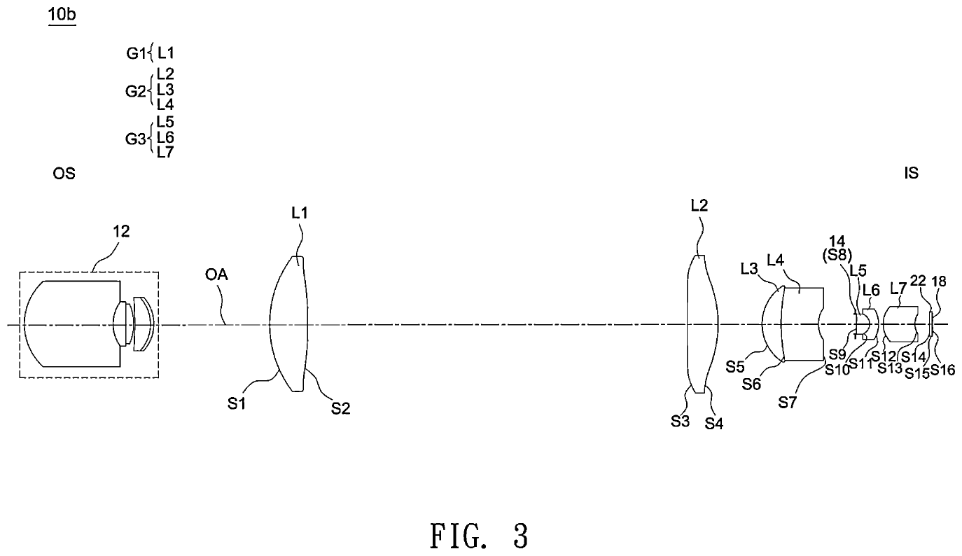

FIG. 3 shows a schematic diagram of an ophthalmic lens according to a second embodiment of the invention. Referring to FIG. 3, the ophthalmic lens 10b includes a lens L1, a lens L2, a lens L3, a lens L4, a lens L5, a lens L6 and a lens L7 in order from the object side OS to the image side IS, an aperture stop 14 is disposed between the lens L4 and the lens L5, and refractive powers of the lenses L1-L7 are respectively positive, positive, positive, negative, positive, negative and positive. In this embodiment, the lens L1, the lens L2 and the lens L7 are plastic aspheric lenses, and the lens L3, the lens L4, the lens L5, and the lens L6 are glass spherical lenses. In this embodiment, the lens L3 and the lens L4 of the lens group G2 form a cemented doublet, and the lens L5 and the lens L6 of the lens group G3 form another cemented doublet, but the invention is not limited to this configuration. According to this embodiment, two cemented doublets are used to minimize the focus shift, ensuring that the discrepancy in the focal planes for visible light and near-infrared light is less than 10 micrometers to thus achieve confocal effects.

In this embodiment of the ophthalmic lens 10b, L1=82.1 mm, LT=136.5 mm, L1/LT=0.60, EFL=−5.342 mm, EFL1=30.09 mm, |LT/EFL|=25.55, |EFL1/EFL|=5.633, R1/R2=0.98, D1=27.72 mm, D2=28.26 mm, D1/D2=0.981, DFOV=45 degrees, and F #=2.0.

Detailed optical data and design parameters of the ophthalmic lens 10b are shown in Table 3, and table 4 lists aspheric coefficients and conic constant of each aspheric surface of the ophthalmic lens 10b.

TABLE 3

| Object | | Radius of | Interval | Refractive | Abbe |
|---|---|---|---|---|---|
| description | Surface | curvature(mm) | (mm) | index (nd) | number (Vd) |
| eye model 12 | | | 25 | | |
| lens L1(aspheric) | S1* | 15.42 | 7.743 | 1.54 | 56.0 |
| | S2* | 208.68 | 82.142 | | |
| lens L2(aspheric) | S3* | −250.00 | 6.353 | 1.64 | 22.4 |
| | S4* | −20.75 | 9.132 | | |
| lens L3(meniscus) | S5 | 9.61 | 3.811 | 1.59 | 35.3 |
| lens L4(meniscus) | S6 | 39.41 | 7.475 | 1.99 | 16.5 |
| | S7 | 4.30 | 7.747 | | |
| aperture stop 14 | S8 | Inf. | 0.129 | | |
| lens L5 | S9 | Inf. | 2.749 | 1.50 | 81.6 |
| (planar-convex) | | | | | |
| lens L6(meniscus) | S10 | −2.68 | 1.959 | 1.99 | 16.5 |
| | S11 | −4.38 | 0.917 | | |
| lens L7(aspheric) | S12* | 4.91 | 6.315 | 1.54 | 56.0 |
| | S13* | 4.23 | 3.061 | | |
| filter 22 | S14 | Inf. | 0.210 | 1.52 | 64.2 |
| | S15 | Inf. | 0.145 | | |
| image plane 18 | S16 | Inf. | 0.000 | | |

TABLE 4

| Surface | S1* | S2* | S3* | S4* | S12* | S13* |
|---|---|---|---|---|---|---|
| K | −0.27 | 0.00 | 0.00 | 0.00 | 0.00 | 0.00 |
| A | −2.30E−04 | −2.89E−04 | 4.64E−05 | 6.56E−05 | −3.79E−04 | 3.84E−03 |
| B | 2.67E−06 | 5.02E−06 | 2.73E−07 | 2.16E−07 | −1.50E−05 | 4.22E−04 |
| C | −2.57E−08 | −6.33E−08 | −5.33E−09 | −3.99E−09 | −7.77E−07 | −3.44E−05 |
| D | 1.81E−10 | 5.79E−10 | 5.79E−11 | 4.78E−11 | −1.47E−08 | −1.26E−06 |
| E | −9.37E−13 | −3.48E−12 | −3.61E−13 | −3.24E−13 | −7.90E−10 | −6.93E−09 |
| F | 3.13E−15 | 1.21E−14 | 1.20E−15 | 1.14E−15 | 8.23E−20 | −1.52E−11 |
| G | −4.73E−18 | −1.78E−17 | −1.66E−18 | −1.67E−18 | 0.00E+00 | −1.51E−15 |

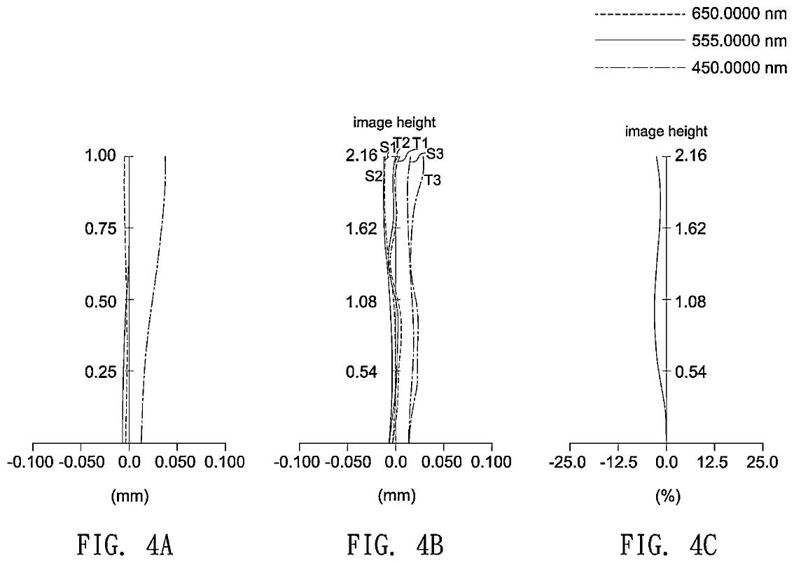

FIG. 4A, FIG. 4B and FIG. 4C show longitudinal spherical aberration curves, astigmatic field curves and a distortion curve of the ophthalmic lens 10b measured at wavelengths of 650 nm, 555 nm, and 450 nm. The simulation results shown in FIGS. 4A-4C are within permitted ranges specified by the standard, which indicates the ophthalmic lens 10b may provide good imaging quality.

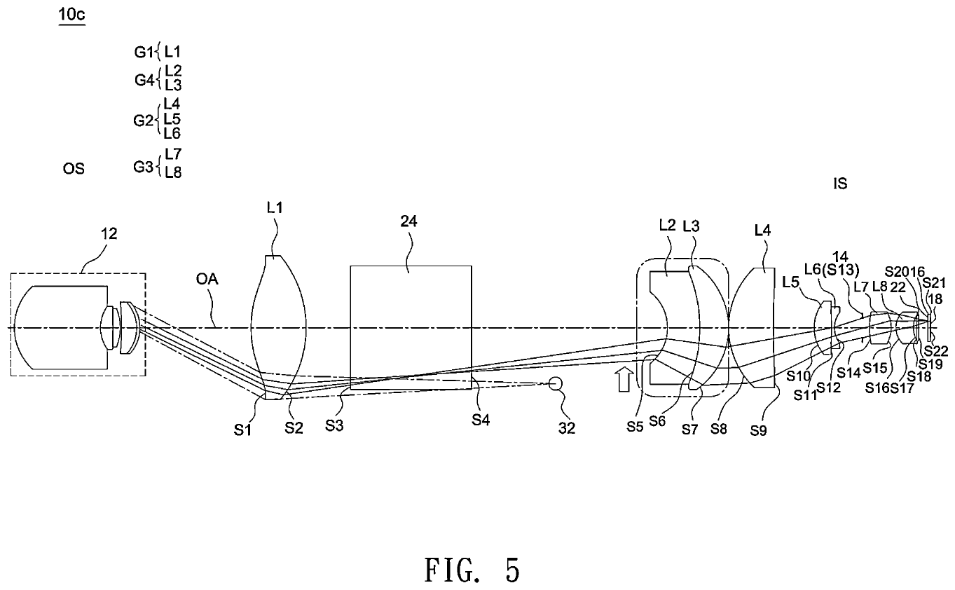

FIG. 5 shows a schematic diagram of an ophthalmic lens according to a third embodiment of the invention. Referring to FIG. 5, the ophthalmic lens 10c includes a lens L1, a lens L2, a lens L3, a lens L4, a lens L5, a lens L6, a lens L7 and a lens L8 in order from the object side OS to the image side IS, an aperture stop 14 is disposed between the lens L6 and the lens L7, and refractive powers of the lenses L1-L8 are respectively positive, negative, positive, positive, positive, negative, positive and positive. In this embodiment, the lens L1, the lens L4 and the lens L8 are plastic aspheric lenses, and the lens L2, the lens L3, the lens L5, the lens L6 and the lens L7 are glass spherical lenses. In this embodiment, the lens L2 and the lens L3 form a cemented doublet, and the lens L5 and the lens L6 form another cemented doublet, but the invention is not limited to this configuration. As shown in FIG. 5, in this embodiment, an afocal lens group G4 consisting of the lens L2 and the lens L3 is added between the lens group G1 and the lens group G2, and the lens L2 and the lens L3 may be cemented together to form a doublet lens. As indicated by the arrow shown in FIG. 5, the afocal lens group G4 allows for contraction of imaging optical paths, thus enhancing the freedom of the arrangement of illumination optical paths and the placement of the illumination source 32. For example, in one embodiment, the illumination source 32 can be positioned closer to lens L1 and within the focal range of lens L1 to reduce light loss during the transmission of illumination light. Furthermore, the afocal lens group G4 can be used to adjust imaging optical paths to ensure the illumination and imaging optical paths are optimally separated to avoid that the ineffective illumination light not entering the ophthalmic is deflected by lenses to become ghosting or stray light.

In this embodiment of the ophthalmic lens 10c, L1=74.39 mm, LT=136.5 mm, L1/LT=0.55, EFL=−5.81 mm, EFL1=25.00 mm, |LT/EFL|=23.47, |EFL1/EFL|=4.303, R1/R2=1.20, D1=28.22 mm, D2=23.46 mm, D1/D2=1.203, DFOV=45 degrees, and F #=2.0.

Detailed optical data and design parameters of the ophthalmic lens 10c are shown in Table 5, and table 6 lists aspheric coefficients and conic constant of each aspheric surface of the ophthalmic lens 10c.

TABLE 5

| | | Radius of | Interval | Refractive | Abbe |
|---|---|---|---|---|---|
| Object description | Surface | curvature(mm) | (mm) | index (nd) | number (Vd) |
| eye model 12 | | | 25 | | |
| lens L1(aspheric) | S1* | 27.99 | 11.500 | 1.54 | 56.0 |
| | S2* | −22.75 | 9.000 | | |
| deflecting element | S3 | Inf. | 25.000 | 1.52 | 64.2 |
| 24 | | | | | |
| | S4 | Inf. | 40.393 | | |
| lens L2(meniscus) | S5 | −9.76 | 6.496 | 1.81 | 22.8 |
| lens L3(meniscus) | S6 | −35.79 | 6.000 | 1.83 | 42.7 |
| | S7 | −16.24 | 0.050 | | |
| lens L4(aspheric) | S8* | 18.39 | 9.326 | 1.64 | 22.4 |
| | S9* | −230.07 | 8.306 | | |
| lens L5(bi-convex) | S10 | 8.03 | 3.602 | 1.83 | 42.7 |
| lens L6(bi-concave) | S11 | −82.35 | 0.522 | 1.99 | 16.5 |
| | S12 | 4.44 | 5.867 | | |
| aperture stop 14 | S13 | Inf. | 1.498 | | |
| lens L7(bi-convex) | S14 | 18.49 | 4.431 | 1.50 | 81.6 |
| | S15 | −7.05 | 0.929 | | |
| lens L8(aspheric) | S16* | 4.37 | 3.545 | 1.54 | 56.0 |
| | S17* | 3.29 | 0.665 | | |
| filter 22 | S18 | Inf. | 0.210 | 1.52 | 64.2 |
| | S19 | Inf. | 2.000 | | |
| cover plate 16 | S20 | Inf. | 0.500 | 1.52 | 64.2 |
| | S21 | Inf. | 0.145 | | |
| image plane 18 | S22 | Inf. | 0.000 | | |

TABLE 6

| Surface | S1* | S2* | S8* | S9* | S16* | S17* |
|---|---|---|---|---|---|---|
| K | 2.79 | 0.00 | 0.00 | 0.00 | 0.00 | 0.71 |
| A | 2.10E−05 | 6.80E−05 | 5.68E−07 | 3.80E−05 | 1.59E−04 | 4.31E−03 |
| B | −4.07E−07 | −2.68E−07 | 4.97E−08 | −3.10E−08 | −4.30E−05 | −2.22E−04 |
| C | −2.56E−09 | −2.66E−09 | −2.69E−09 | −3.31E−09 | 3.50E−06 | −3.64E−05 |
| D | 5.52E−11 | 3.45E−11 | 5.85E−11 | 1.46E−10 | −4.55E−07 | 3.70E−05 |
| E | −4.77E−13 | −1.78E−13 | −5.60E−13 | −2.11E−12 | 0.00E+00 | −7.89E−06 |
| F | 2.05E−15 | 4.66E−16 | 2.65E−15 | 1.43E−14 | 0.00E+00 | 0.00E+00 |
| G | −3.81E−18 | −5.40E−19 | −4.65E−18 | −3.54E−17 | 0.00E+00 | 0.00E+00 |

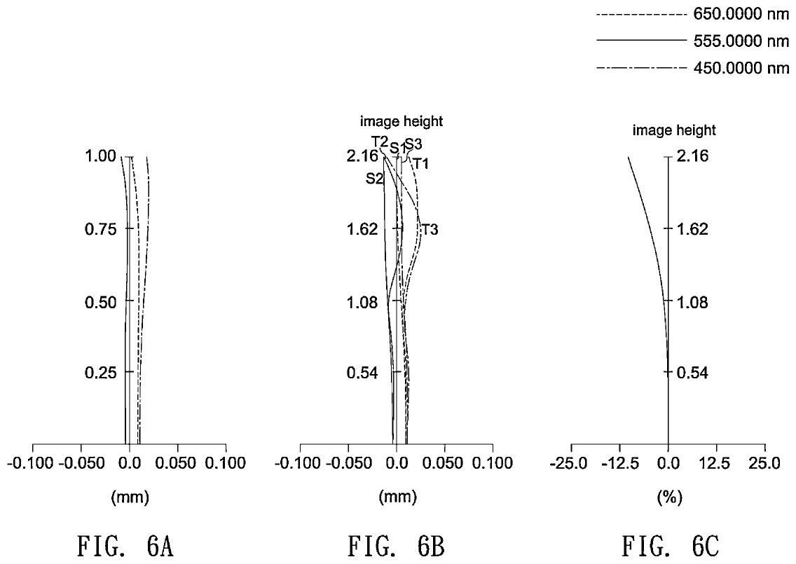

FIG. 6A, FIG. 6B and FIG. 6C show longitudinal spherical aberration curves, astigmatic field curves and a distortion curve of the ophthalmic lens 10c measured at wavelengths of 650 nm, 555 nm, and 450 nm. The simulation results shown in FIGS. 6A-6C are within permitted ranges specified by the standard, which indicates the ophthalmic lens 10c may provide good imaging quality.

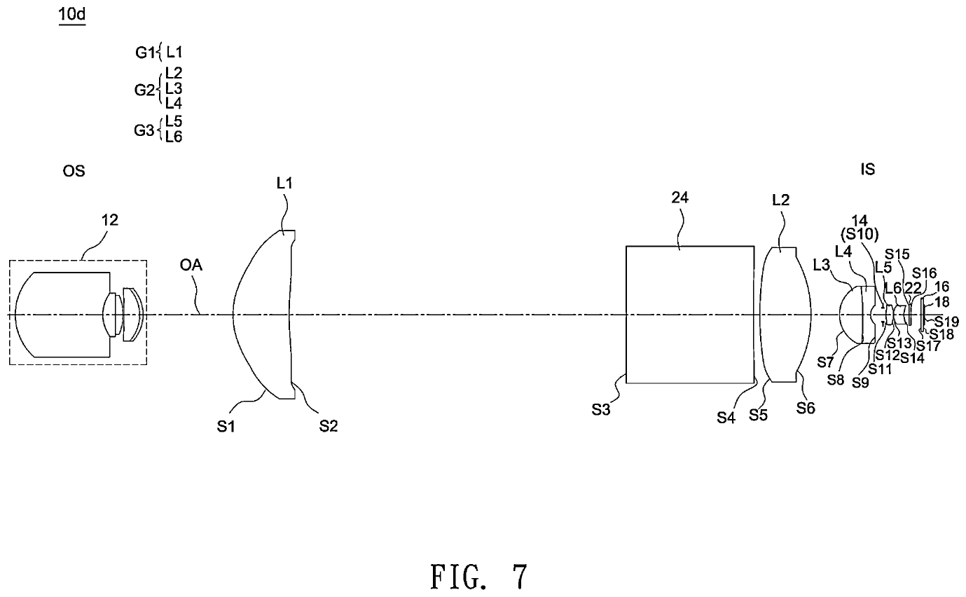

FIG. 7 shows a schematic diagram of an ophthalmic lens according to a fourth embodiment of the invention. Referring to FIG. 7, the ophthalmic lens 10d includes a lens L1, a lens L2, a lens L3, a lens L4, a lens L5 and a lens L6 in order from the object side OS to the image side IS, an aperture stop 14 is disposed between the lens L4 and the lens L5, and refractive powers of the lenses L1-L6 are respectively positive, positive, positive, negative, positive and positive. In this embodiment, the lens L1, the lens L2 and the lens L6 are plastic aspheric lenses, and the lens L3, the lens L4 and the lens L5 are glass spherical lenses. In this embodiment, the lens L3 and the lens L4 form a cemented doublet, but the invention is not limited to this configuration. In this embodiment, the lens L1 can be a gradient-index lens to increase the design freedom of the lens shape. Therefore, the surface shape in the paraxial region of lens L1 can be designed as meniscus or plano-convex, or both the object-side surface and the image-side surface in the paraxial region can be planar.

In this embodiment of the ophthalmic lens 10d, L1=92.39 mm, LT=131.7 mm, L1/LT=0.70, EFL=−4.74 mm, EFL1=27.63 mm, |LT/EFL|=27.80, |EFL1/EFL|=5.829, R1/R2=1.27, D1=31.80 mm, D2=25.08 mm, D1/D2=1.268, DFOV=45 degrees, and F #=2.0.

Detailed optical data and design parameters of the ophthalmic lens 10d are shown in Table 7, and table 8 lists aspheric coefficients and conic constant of each aspheric surface of the ophthalmic lens 10d.

TABLE 7

| | | Radius of | Interval | Refractive | Abbe |
|---|---|---|---|---|---|
| Object description | Surface | curvature(mm) | (mm) | index (nd) | number (Vd) |
| eye model 12 | | | 21.34 | | |
| GRIN lens L1 | S1* | 13.00 | 11.000 | 1.54~1.61 | 49.7~43.6 |
| (aspheric) | | | | | |
| | S2* | 38.12 | 65.995 | | |
| deflecting element | S3 | Inf. | 25.400 | 1.52 | 64.2 |
| 24 | | | | | |
| | S4 | Inf. | 1.000 | | |
| lens L2(aspheric) | S5* | 98.35 | 10.000 | 1.64 | 23.3 |
| | S6* | −16.13 | 5.594 | | |
| lens L3(bi-convex) | S7 | 6.19 | 4.374 | 1.83 | 42.7 |
| lens L4(bi-concave) | S8 | −69.65 | 1.725 | 1.99 | 16.5 |
| | S9 | 1.84 | 2.574 | | |
| aperture stop 14 | S10 | Inf. | 0.553 | | |
| lens L5(bi-convex) | S11 | 37.82 | 1.273 | 1.50 | 81.6 |
| | S12 | −2.98 | 0.050 | | |
| lens L6(aspheric) | S13* | 3.35 | 2.137 | 1.54 | 56.0 |
| | S14* | 3.74 | 1.006 | | |
| filter 22 | S15 | Inf. | 0.210 | 1.52 | 64.2 |
| | S16 | Inf. | 2.000 | | |
| cover plate 16 | S17 | Inf. | 0.500 | 1.52 | 64.2 |
| | S18 | Inf. | 0.145 | | |
| image plane 18 | S19 | Inf. | 0.000 | | |

TABLE 8

| Surface | S1* | S2* | S5* | S6* | S13* | S14* |
|---|---|---|---|---|---|---|
| K | −0.44 | 4.16 | 1.70 | 0.01 | −0.12 | 0.00 |
| A | −9.79E−05 | −1.56E−04 | 6.25E−05 | 1.53E−04 | 1.48E−03 | 8.18E−03 |
| B | −4.39E−08 | 9.25E−07 | −9.11E−07 | −1.44E−06 | 3.09E−05 | 1.34E−03 |
| C | −2.02E−10 | −4.94E−09 | 1.20E−08 | 1.83E−08 | −4.25E−05 | 1.69E−04 |
| D | 5.83E−12 | 1.60E−11 | −1.57E−10 | −1.92E−10 | −3.56E−06 | −1.08E−04 |
| E | −1.55E−14 | −1.51E−14 | 1.57E−12 | 1.76E−12 | 2.66E−11 | −6.93E−09 |
| F | 0.00E+00 | 0.00E+00 | −7.99E−15 | −9.48E−15 | −8.83E−21 | −1.52E−11 |
| G | 0.00E+00 | 0.00E+00 | 1.57E−17 | 2.11E−17 | 0.00E+00 | −1.51E−15 |

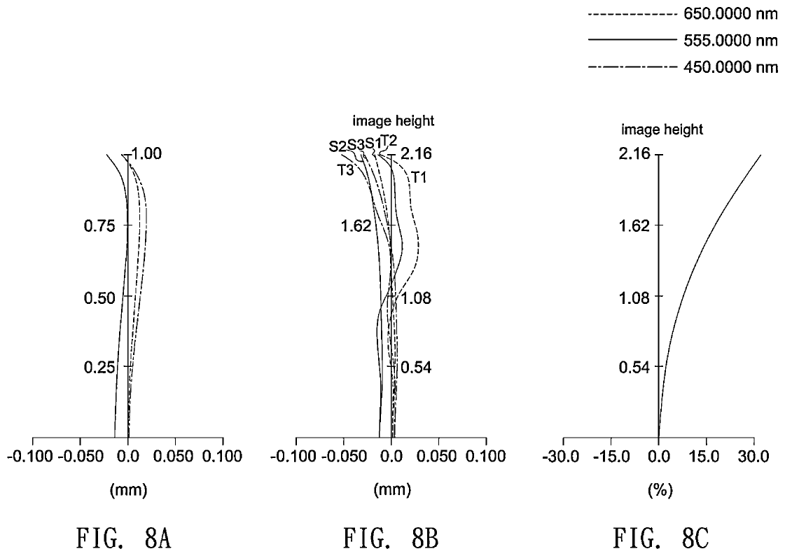

FIG. 8A, FIG. 8B and FIG. 8C show longitudinal spherical aberration curves, astigmatic field curves and a distortion curve of the ophthalmic lens 10d measured at wavelengths of 650 nm, 555 nm, and 450 nm. The simulation results shown in FIGS. 8A-8C are within permitted ranges specified by the standard, which indicates the ophthalmic lens 10d may provide good imaging quality.

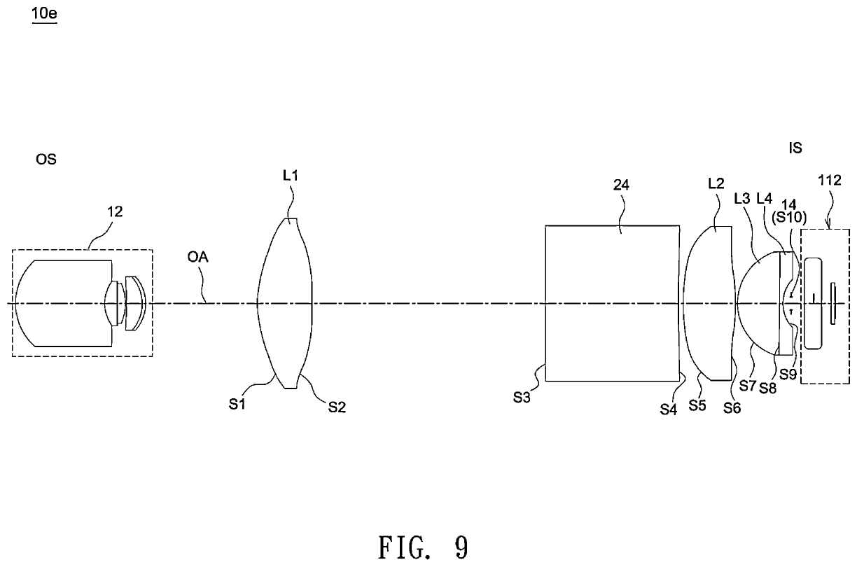

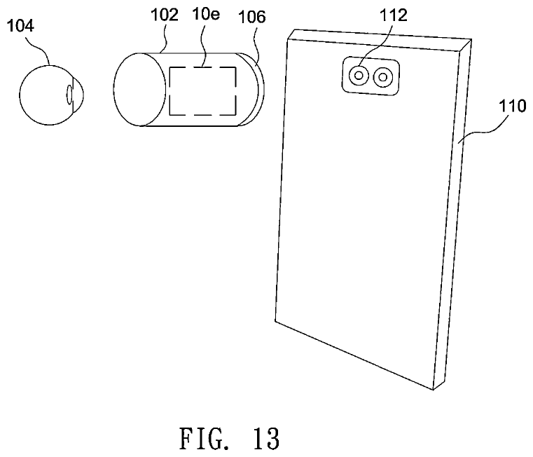

FIG. 9 shows a schematic diagram of an ophthalmic lens according to a fifth embodiment of the invention. In this embodiment, the ophthalmic lens 10e can be used as a relay lens in conjunction with a smartphone for imaging. As shown in FIG. 13, the ophthalmic lens 10e is housed within a casing 102, where one end of the casing 102 is attached to a smart phone 110 via a lens module interface 106, and the other end is configured to point towards the pupil of the human eye 104. The relay ophthalmic lens 10e initially captures light reflected from the fundus to perform primary imaging. This captured image is then transformed into parallel light rays, and finally these parallel rays are focused by the smartphone camera 112 to create the final image, thus achieving the purpose of capturing a fundus photograph of a human eye with the smart phone 110 ready for subsequent processing. The smart phone 110 described in this embodiment is provided merely as an example, and the relay ophthalmic lens 10e can be integrated with other mobile imaging device (such as a tablet) without limitation. As shown in FIG. 9, the relay ophthalmic lens 10e includes, in order from the object side OS to the image side IS, a lens L1, a lens L2, a lens L3 and a lens L4 with refractive powers. The refractive powers of the lenses L1 to L4 are positive, positive, positive and negative, respectively. The lens L1 and the lens L2 are plastic aspheric lenses, the lens L3 and the lens L4 are glass spherical lenses, and the lens L3 and the lens L4 form a cemented doublet, but the invention is not limited to this configuration. The lenses L1-L4 can be so configured that the pupil of a human eye and the exit pupil of an external imaging system (e.g., smartphone camera) form a conjugate relationship, making the ophthalmic lens 10e an afocal lens.

In this embodiment of the ophthalmic lens 10e, L1=71.3 mm, LT=100.8 mm, L1/LTS0.707, EFL=−5.03 mm, EFL1=30.48 mm, |LT/EFL|=20.04, |EFL1/EFL|=6.06, R1/R2=1.1, D1=30.85 mm, D2=27.98 mm, D1/D21.102, DFOV45 degrees, and F #62.0.

Detailed optical data and design parameters of the ophthalmic lens 10e are shown in Table 9, and table 10 lists aspheric coefficients and conic constant of each aspheric surface of the ophthalmic lens 10e.

TABLE 9

| | | Radius of | Interval | Refractive | Abbe |
|---|---|---|---|---|---|
| Object description | Surface | curvature(mm) | (mm) | index (nd) | number (Vd) |
| eye model 12 | | | 21.34 | | |
| L1(aspheric) | S1* | 15.85 | 10.612 | 1.54 | 56.3 |
| | S2* | 250.00 | 44.919 | | |
| deflecting element | S3 | Inf. | 25.400 | 1.52 | 64.2 |
| 24 | | | | | |
| | S4 | Inf. | 1.000 | | |
| lens L2(aspheric) | S5* | 69.13 | 9.842 | 1.64 | 23.3 |
| | S6* | −30.13 | 0.360 | | |
| lens L3(bi-convex) | S7 | 10.19 | 8.211 | 1.88 | 40.8 |
| lens L4(bi-concave) | S8 | −304.33 | 0.500 | 1.99 | 16.5 |
| | S9 | 6.24 | 6.089 | | |
| aperture stop 14 | S10 | Inf. | 0.018 | | |
| smart phone 112 | S11 | Inf. | 0.000 | | |

TABLE 10

| Surface | S1* | S2* | S5* | S6* |
|---|---|---|---|---|
| K | −0.39 | 0.00 | 17.80 | 0.95 |
| A | −1.63E−04 | −2.52E−04 | 1.06E−04 | 1.25E−04 |
| B | 5.45E−07 | 2.59E−06 | −6.39E−07 | −4.52E−07 |
| C | 1.77E−09 | −2.10E−08 | 1.85E−09 | −5.96E−09 |
| D | −3.73E−11 | 1.25E−10 | 1.63E−11 | 1.55E−10 |
| E | 2.01E−13 | −4.95E−13 | −1.67E−13 | −1.34E−12 |
| F | −4.79E−16 | 1.14E−15 | 5.82E−16 | 5.48E−15 |
| G | 4.38E−19 | −1.11E−18 | −7.05E−19 | −8.59E−18 |

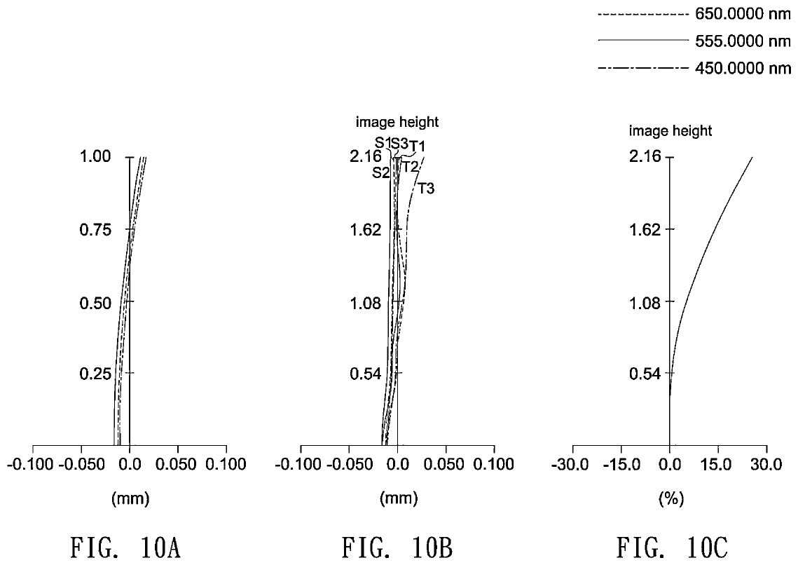

FIG. 10A, FIG. 10B and FIG. 10C show longitudinal spherical aberration curves, astigmatic field curves and a distortion curve of the ophthalmic lens 10e measured at wavelengths of 650 nm, 555 nm, and 450 nm. Since the ophthalmic lens 10e is a relay lens and cannot directly form an image, the simulation results shown in FIGS. 10A to 10C are obtained by combining the ophthalmic lens 10e with an ideal lens (a lens without spherical aberration) with a focal length of 4 mm to focus and form an image, in order to verify the optical performance of the ophthalmic lens 10e. The simulation results shown in FIGS. 10A-10C are within permitted ranges specified by the standard, which indicates the ophthalmic lens 10e may provide good imaging quality.

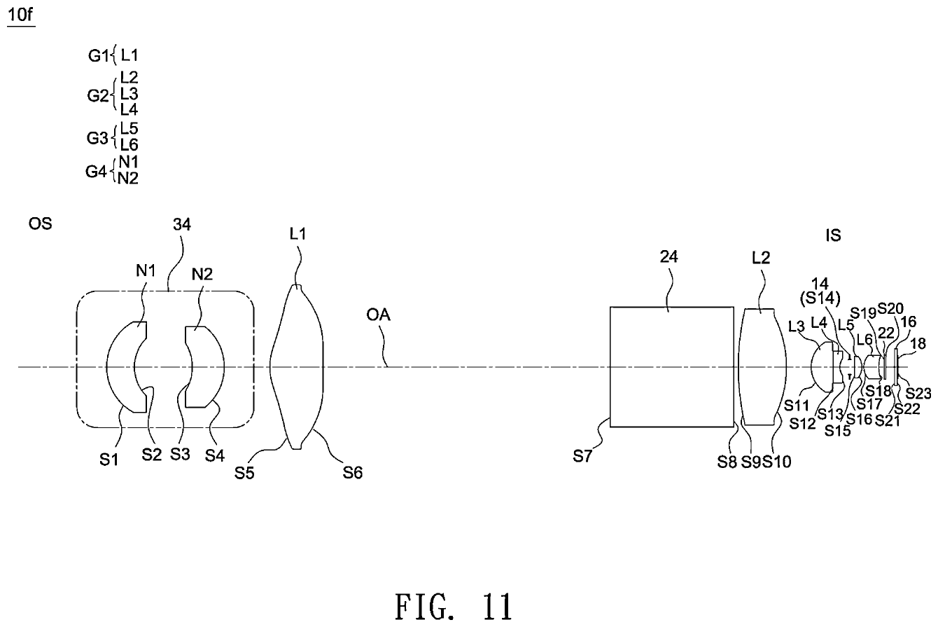

FIG. 11 shows a schematic diagram of an ophthalmic lens according to a sixth embodiment of the invention. In this embodiment, the ophthalmic lens 10f includes a lens L1, a lens L2, a lens L3, a lens L4, a lens L5 and a lens L6 in order from the object side OS to the image side IS, and an aperture stop 14 is disposed between the lens L4 and the lens L5. Furthermore, the lens L1 can be detachably coupled to a lens module 34 via a lens module interface 106 as shown in FIG. 13, for example. The lens module 34 may contain conversion lenses N1 and N2, allowing the ophthalmic lens 10e to have the function of imaging general environments. After removing the lens module 34, the ophthalmic lens 10e can be adapted for use in ophthalmic photography applications. In this embodiment, the lens module 34 includes at least two lenses, and the conversion lens N1 closest to the object side OS is movable to achieve an applicable shooting distance from infinity to 10 cm. Besides, by moving the conversion lens N1, field curvature aberration can be corrected to obtain optimal resolution. Moreover, refractive powers of the lenses L1-L6 are respectively positive, positive, positive, negative, positive and positive. In this embodiment, the lens L1, the lens L2 and the lens L6 are plastic aspheric lenses, and the lens L3, the lens L4 and the lens L5 are glass spherical lenses. In this embodiment, the lens L3 and the lens L4 form a cemented doublet, but the invention is not limited to this configuration.

In this embodiment of the ophthalmic lens 10f, L1=85.8 mm, LT=163.8 mm, L1/LT=0.524, EFL=−4.69 mm, EFL1=27.48 mm, |LT/EFL|=34.925, |EFL1/EFL|=5.859, R1/R2=1.43, D1=31.84 mm, D2=22.27 mm, D1/D2=1.430, DFOV=45 degrees, and F #=2.0.

Detailed optical data and design parameters of the ophthalmic lens 10f are shown in Table 11, and table 12 lists aspheric coefficients and conic constant of each aspheric surface of the ophthalmic lens 10f.

TABLE 11

| | | Radius of | Interval | Refractive | Abbe |
|---|---|---|---|---|---|
| Object description | Surface | curvature(mm) | (mm) | index (nd) | number (Vd) |
| conversion lens N1 | S1 | 10.64 | 5.545 | 1.83 | 42.7 |
| | S2 | 8.59 | 12.227 | | |
| conversion lens N2 | S3 | −8.31 | 6.495 | 1.70 | 55.5 |
| | S4 | −10.41 | 9.501 | | |
| L1(aspheric) | S5* | 14.38 | 11.000 | 1.54 | 56.3 |
| | S6* | 250.00 | 59.443 | | |
| deflecting element | S7 | Inf. | 25.400 | 1.52 | 64.2 |
| 24 | | | | | |
| | S8 | Inf. | 1.000 | | |
| lens L2(aspheric) | S9* | 92.15 | 9.999 | 1.64 | 23.3 |
| | S10* | −16.24 | 5.262 | | |
| lens L3(meniscus) | S11 | 5.85 | 4.388 | 1.83 | 42.7 |
| lens L4(meniscus) | S12 | 71.52 | 1.290 | 1.99 | 16.5 |
| | S13 | 2.00 | 2.435 | | |
| aperture stop 14 | S14 | Inf. | 0.918 | | |
| lens L5(meniscus) | S15 | −37.09 | 1.436 | 1.50 | 81.6 |
| | S16 | −3.19 | 0.554 | | |
| lens L6(aspheric) | S17* | 4.40 | 3.000 | 1.54 | 56.3 |
| | S18* | 8.72 | 1.146 | | |
| filter 22 | S19 | Inf. | 0.210 | 1.52 | 64.2 |
| | S20 | Inf. | 2.000 | | |
| cover plate 16 | S21 | | 0.500 | 1.52 | 64.2 |
| | S22 | Inf. | 0.045 | | |
| image plane 18 | S23 | Inf. | 0.000 | | |

TABLE 12

| Surface | S5* | S6* | S9* | S10* | S17* | S18* |
|---|---|---|---|---|---|---|
| K | −0.48 | 0.00 | 2.42 | 0.00 | −0.34 | 0.00 |
| A | −1.66E−04 | −2.43E−04 | 5.70E−05 | 1.48E−04 | 1.06E−03 | 2.99E−03 |
| B | 3.35E−07 | 2.18E−06 | −5.45E−07 | −9.82E−07 | 3.00E−05 | 6.98E−04 |
| C | −1.34E−09 | −1.82E−08 | 2.62E−09 | 4.77E−09 | −9.34E−06 | −5.89E−05 |
| D | 1.08E−11 | 1.09E−10 | 5.27E−12 | 5.06E−11 | 2.60E−07 | −1.17E−07 |
| E | −5.53E−14 | −4.19E−13 | −1.07E−13 | −8.65E−13 | 2.66E−11 | −6.93E−09 |
| F | 1.54E−16 | 9.27E−16 | 5.79E−16 | 5.06E−15 | −8.83E−21 | −1.52E−11 |
| G | −1.82E−19 | −8.74E−19 | −1.03E−18 | −1.01E−17 | 0.00E+00 | −1.51E−15 |

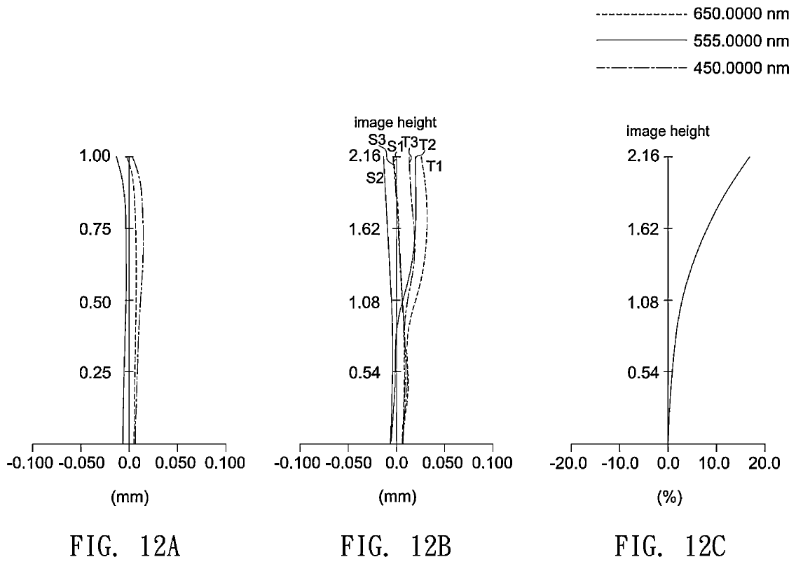

FIG. 12A, FIG. 12B and FIG. 12C show longitudinal spherical aberration curves, astigmatic field curves and a distortion curve of the ophthalmic lens 10f measured at wavelengths of 650 nm, 555 nm, and 450 nm. The simulation results shown in FIGS. 12A-12C are within permitted ranges specified by the standard, which indicates the ophthalmic lens 10f may provide good imaging quality.

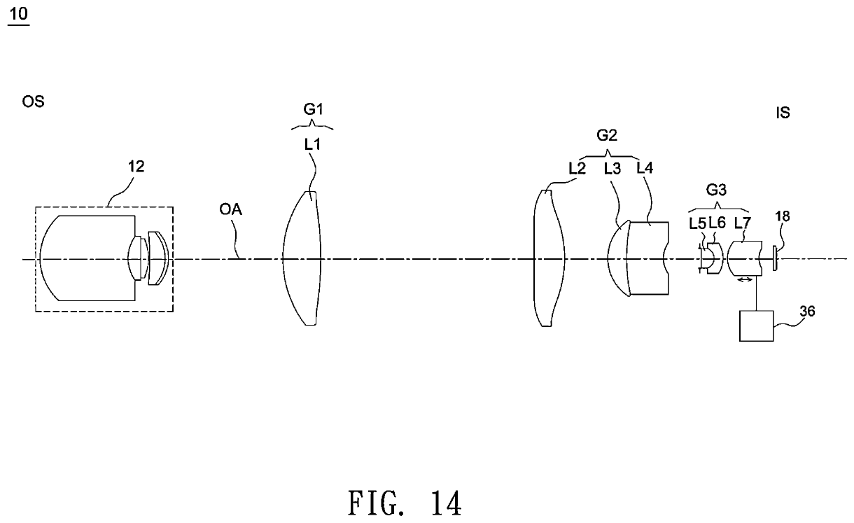

FIG. 14 shows a schematic diagram of an ophthalmic lens according to another embodiment of the invention. As shown in FIG. 14, the ophthalmic lens 10 may include a diopter adjustment mechanism 36. In this embodiment, the diopter adjustment mechanism 36 is located at a position near the lens group G3 that is closest to the image side IS, and the diopter adjustment mechanism 36 may move at least one lens of the lens group G3, such as moving the lens closest to the image side IS (lens L7), to adjust the diopter of the ophthalmic lens 10. Because the diopter adjustment mechanism 36 is allowed to provide the ophthalmic lens with a wider diopter range, a clear ophthalmic photograph for subjects with severe myopia (nearsightedness) or severe hyperopia (farsightedness) can be still obtained.

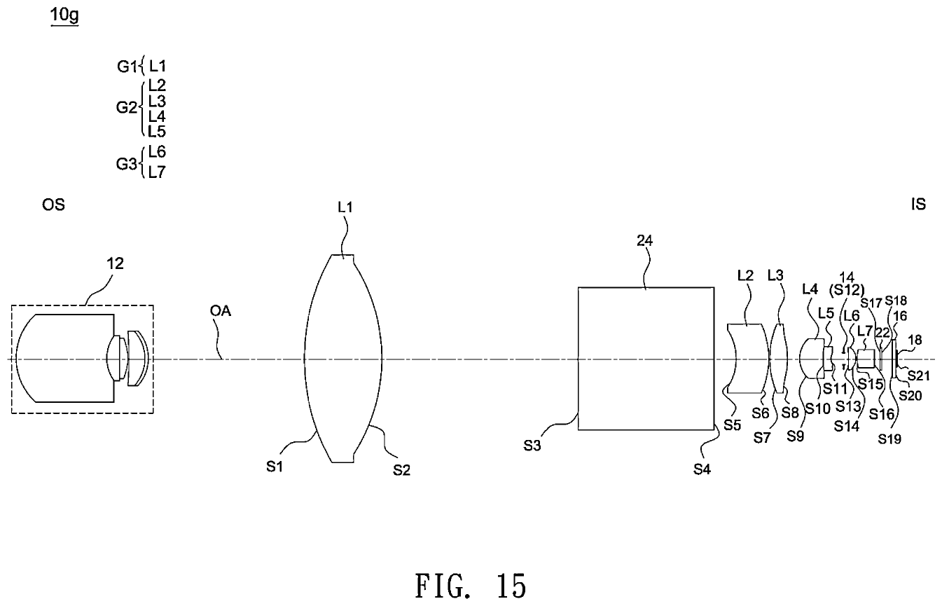

FIG. 15 shows a schematic diagram of an ophthalmic lens according to another embodiment of the invention. Referring to FIG. 15, the ophthalmic lens 10g includes a lens group G1, a lens group G2 and a lens group G3 arranged in order along a direction, the lens group G1 includes a lens L1, the lens group G2 includes a lens L2, a lens L3, a lens L4 and a lens L5, and the lens group G3 includes a lens L6 and a lens L7. In this embodiment, the ophthalmic lens 10g is housed within a barrel (not shown) and consists essentially of seven lenses, namely the lenses L1, L2, L3, L4, L5, L6 and L7 arranged in order from an object side OS to an image side IS of the ophthalmic lens 10g, where the object side OS corresponds to the position of an eye model 12 and the image side IS corresponds to the position of an image plane 18 of an image sensor. In this embodiment, the refractive powers of the lenses L1 to L7 along the optical axis OA are respectively positive, negative, positive, positive, negative, negative and positive. The lens L1 is a glass-molded aspheric lens, the lens L2, the lens L3 and the lens L7 are plastic aspheric lenses, and the lens L4, the lens L5, and the lens L6 are glass spherical lenses, but the invention is not limited thereto. In this embodiment, the lens L4 and the lens L5 form a cemented doublet, but the invention is not limited to this configuration. In this embodiment of the ophthalmic lens 10g, L1=65.7 mm, LT=110.1 mm, L1/LT=0.60, EFL=−5.18 mm, EFL1=24.31 mm, |LT/EFL|=21.25, |EFL1/EFL|=4.69, R1/R2=3.13, D1=37.6 mm, D2=12.7 mm, D1/D2=2.96, DFOV=45 degrees, and F #=3.0.

Detailed optical data and design parameters of the ophthalmic lens 10g are shown in Table 13, and table 14 lists aspheric coefficients and conic constant of each aspheric surface of the ophthalmic lens 10g.

TABLE 13

| | | Radius of | Interval | Refractive | Abbe |
|---|---|---|---|---|---|
| Object description | Surface | curvature(mm) | (mm) | index (nd) | number (Vd) |
| eye model 12 | | | 29.34 | | |
| lens L1(aspheric) | S1* | 32.62 | 14.520 | 1.69 | 53.2 |
| | S2* | −28.74 | 36.318 | | |
| deflecting element | S3 | Inf. | 25.400 | 1.52 | 64.2 |
| 24 | | | | | |
| | S4 | Inf. | 4.000 | | |
| lens L2(aspheric) | S5* | −11.01 | 5.999 | 1.54 | 56.3 |
| | S6* | −15.32 | 0.200 | | |
| lens L3(aspheric) | S7* | 35.12 | 3.192 | 1.64 | 23.3 |
| | S8* | −16.32 | 2.269 | | |
| lens L4(meniscus) | S9 | 5.85 | 4.388 | 1.83 | 42.7 |
| lens L5(meniscus) | S10 | 71.52 | 1.290 | 1.99 | 16.5 |
| | S11 | 2.00 | 2.435 | | |
| aperture stop 14 | S12 | Inf. | 0.918 | | |
| lens L6(meniscus) | S13 | −37.09 | 1.436 | 1.50 | 81.6 |
| | S14 | −3.19 | 0.050 | | |
| lens L7(aspheric) | S15* | 6.98 | 3.427 | 1.54 | 56.3 |
| | S16* | −52.66 | 1.000 | | |
| filter 22 | S17 | Inf. | 0.210 | 1.52 | 64.2 |
| | S18 | Inf. | 2.234 | | |
| cover plate 16 | S19 | Inf. | 0.500 | 1.52 | 64.2 |
| | S20 | Inf. | 0.145 | | |
| image plane 18 | S21 | Inf. | 0.000 | | |

TABLE 14

| Surface | S1* | S2* | S5* | S6* | S7* | S8* |
|---|---|---|---|---|---|---|
| K | −7.66 | 0.00 | 0.59 | 0.00 | 0.00 | −0.37 |
| A | 0.00E+00 | 3.16E−06 | −1.49E−04 | 1.18E−04 | 2.88E−04 | 2.19E−04 |
| B | −2.02E−08 | 9.25E−09 | 7.50E−06 | 2.66E−07 | 1.52E−06 | 3.98E−06 |
| C | 1.50E−10 | 8.99E−11 | −4.19E−08 | −4.36E−08 | −2.90E−07 | −3.76E−07 |
| D | −3.04E−13 | −3.19E−13 | −1.03E−09 | 2.26E−10 | 1.15E−08 | 1.71E−08 |
| E | 4.17E−16 | 6.26E−16 | 0.00E+00 | 0.00E+00 | −2.15E−10 | −3.66E−10 |
| F | 0.00E+00 | 0.00E+00 | 0.00E+00 | 0.00E+00 | 1.95E−12 | 3.56E−12 |
| G | 0.00E+00 | 0.00E+00 | 0.00E+00 | 0.00E+00 | 0.00E+00 | 0.00E+00 |

| Surface | S15* | S16* |
|---|---|---|
| K | −1.01 | 0.00 |
| A | 7.81E−05 | 1.16E−04 |
| B | −1.05E−04 | −1.39E−04 |
| C | 9.16E−06 | 2.57E−05 |
| D | −2.41E−06 | −3.98E−06 |
| E | 0.00E+00 | 0.00E+00 |
| F | 0.00E+00 | 0.00E+00 |
| G | 0.00E+00 | 0.00E+00 |

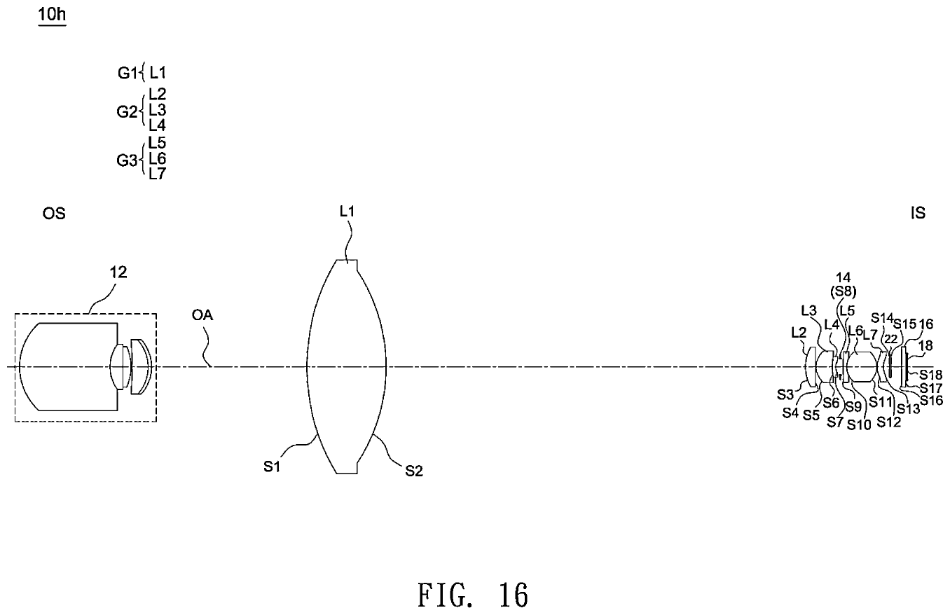

FIG. 16 shows a schematic diagram of an ophthalmic lens according to another embodiment of the invention. Referring to FIG. 16, the ophthalmic lens 10h includes a lens group G1, a lens group G2 and a lens group G3 arranged in order along a direction, the lens group G1 includes a lens L1, the lens group G2 includes a lens L2, a lens L3 and a lens L4, and the lens group G3 includes a lens L5, a lens L6 and a lens L7. In this embodiment, the ophthalmic lens 10h is housed within a barrel (not shown) and consists essentially of seven lenses, namely the lenses L1, L2, L3, L4, L5, L6 and L7 arranged in order from an object side OS to an image side IS of the ophthalmic lens 10h, where the object side OS corresponds to the position of an eye model 12 and the image side IS corresponds to the position of an image plane 18 of an image sensor. In this embodiment, the refractive powers of the lenses L1 to L7 along the optical axis OA are respectively positive, positive, positive, negative, negative positive and positive. The lens L1 is a glass-molded aspheric lens, the lens L2 and the lens L7 are plastic aspheric lenses, and the lens L3, the lens L4, the lens L5 and the lens L6 are glass spherical lenses, but the invention is not limited thereto. In this embodiment, the lens L3 and the lens L4 form a cemented doublet, the lens L5 and the lens L6 form another cemented doublet, but the invention is not limited to this configuration. In this embodiment of the ophthalmic lens 10h, L1=78.6 mm, LT=111.6 mm, L1/LT=0.70, EFL=−5.26 mm, EFL1=24.30 mm, |LT/EFL|=21.21, |EFL1/EFL|=4.62, R1/R2=5.12, D1=37.6 mm, D2=7.4, D1/D2=5.06, DFOV=45.4 degrees, and F #=2.0.

Detailed optical data and design parameters of the ophthalmic lens 10h are shown in Table 15, and table 16 lists aspheric coefficients and conic constant of each aspheric surface of the ophthalmic lens 10h.

TABLE 15

| | | Radius of | Interval | Refractive | Abbe |
|---|---|---|---|---|---|
| Object description | Surface | curvature(mm) | (mm) | index (nd) | number (Vd) |
| eye model 12 | | | 29.34 | | |
| lens L1(aspheric) | S1* | 32.62 | 14.520 | 1.69 | 53.2 |
| | S2* | −28.74 | 78.600 | | |
| lens L2(aspheric) | S3* | 7.75 | 2.000 | 1.64 | 23.3 |
| | S4* | −47.45 | 0.100 | | |
| lens L3(meniscus) | S5 | 4.84 | 2.919 | 1.83 | 42.7 |
| lens L4(meniscus) | S6 | 18.98 | 0.500 | 1.99 | 16.5 |
| | S7 | 2.25 | 0.539 | | |
| aperture stop 14 | S8 | Inf. | 0.992 | | |
| lens L5(bi-concave) | S9 | −79.19 | 0.500 | 1.81 | 25.5 |
| lens L6(bi-convex) | S10 | 4.08 | 5.409 | 1.62 | 63.4 |
| | S11 | −4.08 | 0.100 | | |
| lens L7(aspheric) | S12* | 4.86 | 1.402 | 1.54 | 56.3 |
| | S13* | 4.88 | 1.000 | | |
| filter 22 | S14 | Inf. | 0.210 | 1.52 | 64.2 |
| | S15 | Inf. | 2.136 | | |
| cover plate 16 | S16 | Inf. | 0.500 | 1.52 | 64.2 |
| | S17 | Inf. | 0.145 | | |
| image plane 18 | S18 | Inf. | 0.000 | | |

TABLE 16

| Surface | S1* | S2* | S3* | S4* | S12* | S13* |
|---|---|---|---|---|---|---|
| K | −7.66 | 0.00 | 0.00 | 0.00 | 0.00 | 0.00 |
| A | 0.00E+00 | 3.16E−06 | −6.51E−06 | 3.14E−04 | −3.32E−03 | −6.15E−03 |
| B | −2.02E−08 | 9.25E−09 | −1.16E−06 | −1.08E−05 | −2.27E−04 | −3.72E−04 |
| C | 1.50E−10 | 8.99E−11 | −1.01E−07 | 8.31E−07 | −4.49E−06 | −1.33E−05 |
| D | −3.04E−13 | −3.19E−13 | 2.64E−08 | 0.00E+00 | −1.13E−06 | 1.41E−06 |
| E | 4.17E−16 | 6.26E−16 | 0.00E+00 | 0.00E+00 | 0.00E+00 | 0.00E+00 |
| F | 0.00E+00 | 0.00E+00 | 0.00E+00 | 0.00E+00 | 0.00E+00 | 0.00E+00 |
| G | 0.00E+00 | 0.00E+00 | 0.00E+00 | 0.00E+00 | 0.00E+00 | 0.00E+00 |

Through the designs of various embodiments of the invention, an ophthalmic lens with at least one of the advantages of a wide field of view, low ghost imaging, a large aperture, a reduced number of lenses, a wide diopter range, and high resolution visible/near-infrared imaging can be provided. Additionally, a high imaging quality ophthalmic lens that can be coupled with a mobile device or other lens module is also provided.

Though the embodiments of the invention have been presented for purposes of illustration and description, they are not intended to be exhaustive or to limit the invention. Accordingly, many modifications and variations without departing from the spirit of the invention or essential characteristics thereof will be apparent to practitioners skilled in this art. It is intended that the scope of the invention be defined by the claims appended hereto and their equivalents in which all terms are meant in their broadest reasonable sense unless otherwise indicated.

What is claimed is:

1. An ophthalmic lens, comprising:
a first lens group having a positive refractive power and including a first lens, and the first lens being an aspheric lens;
a second lens group having a positive refractive power and including three lenses, one of the three lenses being an aspheric lens, a lens in the second lens group closest to the first lens group being a second lens, and the three lenses including two closely adjacent surfaces having substantially the same curvatures;
a third lens group having a positive refractive power and including two lenses, and one of the two lenses being an aspheric lens; and
an aperture stop disposed between the second lens group and the third lens group;
wherein a total number of lenses with refractive powers of the ophthalmic lens ranges from 6 to 9, and the ophthalmic lens satisfies the following conditions:
0.36<L1/LT<1.12, where L1 is a distance between the first lens group and the second lens group measured along an optical axis of the ophthalmic lens, and LT is a distance measured along the optical axis between two outermost lens surfaces at opposite ends of the ophthalmic lens;
0.65<R1/R2<7.69, where R1 is a shortest distance from a central axis of an optical surface of the first lens to an outermost turning point of the optical surface of the first lens, and R2 is a shortest distance from a central axis of an optical surface of the second lens to an outermost turning point of the optical surface of the second lens; and
14.18<|LT/EFL|<41.69, where EFL is an effective focal length of the ophthalmic lens.

2. The ophthalmic lens as claimed in claim 1, wherein a distance between the first lens group and the second lens group is greater than a distance between the second lens group and the third lens group along the optical axis of the ophthalmic lens.

3. The ophthalmic lens as claimed in claim 1, wherein the ophthalmic lens further comprises a diopter adjustment mechanism located at a position near the third lens group, and the diopter adjustment is capable of moving at least one lens of the third lens group to adjust a diopter of the ophthalmic lens.

4. The ophthalmic lens as claimed in claim 1, wherein the third lens group comprises a cemented lens.

5. The ophthalmic lens as claimed in claim 1, wherein a distance between the first lens group and the second lens group is more than twice a length of the second lens group measured along the optical axis of the ophthalmic lens.

6. The ophthalmic lens as claimed in claim 1, further comprising a light deflecting element arranged between the first lens group and the second lens group in an illumination optical path of the ophthalmic lens.

7. The ophthalmic lens as claimed in claim 6, wherein the light deflecting element is a polarizing beam splitter or a dichroic mirror.

8. The ophthalmic lens as claimed in claim 1, wherein the second lens group further includes an afocal lens group, and the afocal lens group consists of two cemented glass lenses.

9. The ophthalmic lens as claimed in claim 1, wherein a lens closest to an image side of the ophthalmic lens has a concave surface in a paraxial region, and the concave surface faces the image side.

10. The ophthalmic lens as claimed in claim 1, wherein a lens closest to an object side of the ophthalmic lens is a plano-convex lens or a meniscus lens, and a convex surface in a paraxial region of the lens closest to the object side faces the object side.

11. The ophthalmic lens as claimed in claim 1, wherein the ophthalmic lens satisfies a condition of 4.3<|EFL1/EFL|<5.832, where EFL1 is an effective focal length of the first lens group, and EFL is the effective focal length of the ophthalmic lens.

12. The ophthalmic lens as claimed in claim 1, wherein the ophthalmic lens satisfies a condition of 0.65<D1/D2<2.42, where D1 is a diameter of an optical surface of the first lens, and D2 is a diameter of an optical surface of the second lens.

13. The ophthalmic lens as claimed in claim 1, wherein an F-number of the ophthalmic lens is greater than 1.6 and less than 2.8.

14. The ophthalmic lens as claimed in claim 1, wherein a diagonal field of view of the ophthalmic lens is greater than 30 degrees and less than 60 degrees.

15. The ophthalmic lens as claimed in claim 1, wherein the ophthalmic lens further satisfies conditions of 0.55<L1/LT<0.75, 0.98<R1/R2<2.42, and 21.27<|LT/EFL|<27.80.

16. An ophthalmic lens, comprising:
a first lens group having a positive refractive power and including a first lens, and the first lens being an aspheric lens;
a second lens group having a positive refractive power and including three lenses, and the three lenses including an aspheric lens and a cemented lens; and
a lens module interface for coupling the ophthalmic lens with a lens module or a smartphone;
wherein the ophthalmic lens satisfies the following condition:
0.65<R1/R2<7.69, where R1 is a shortest distance from a central axis of an optical surface of the first lens to an outermost turning point of the optical surface of the first lens, and R2 is a shortest distance from a central axis of an optical surface of the second lens to an outermost turning point of the optical surface of the second lens.

17. The ophthalmic lens as claimed in claim 16, wherein a distance between the first lens group and the second lens group is more than twice a length of the second lens group measured along the optical axis of the ophthalmic lens.

18. The ophthalmic lens as claimed in claim 16, further comprising a polarizing beam splitter or a dichroic mirror arranged between the first lens group and the second lens group in an illumination optical path of the ophthalmic lens.

19. The ophthalmic lens as claimed in claim 16, wherein the second lens group further includes an afocal lens group, and the afocal lens group consists of two cemented glass lenses.

20. The ophthalmic lens as claimed in claim 16, wherein the ophthalmic lens satisfies a condition of 4.3<|EFL1/EFL|<5.832, where EFL1 is an effective focal length of the first lens group, and EFL is an effective focal length of the ophthalmic lens.
